(19) United States
(12) Patent Application Publication
(10) Pub. No.: US 2025/0160658 A1
(43) Pub. Date: May 22, 2025

(54) CONTROLLING PATIENT MONITORING DEVICES

(71) Applicant: Cilag GmbH International, Zug (CH)

(72) Inventors: Frederick E. Shelton, IV, Hillsboro, OH (US); Jason L. Harris, Lebanon, OH (US)

(21) Appl. No.: 18/810,419

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. A61B 5/0205; A61B 5/021; A61B 5/024; A61B 5/0531

(52) U.S. Cl. CPC A61B 5/0205; A61B 5/0531; A61B 5/021; A61B 5/024; A61B 2560/0276

(57) ABSTRACT

Examples herein may allow healthcare professionals (HCPs) (e.g., surgeons) to be notified about leakage current in surgical devices. In examples, an electronic station within a surgical system may include a communications interface configured to connect to a plurality of patient monitoring devices. The electronic station may detect a leakage current associated with a patient monitoring device of the plurality of patient monitoring devices. The electronic station may determine a mechanism to compensate for the leakage current associated with the patient monitoring device. The electronic station may perform the mechanism to compensate for the leakage current associated with the patient monitoring device.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,

Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

### BACKGROUND

Surgical procedures are typically performed in surgical operating theaters or rooms in a healthcare facility such as, for example, a hospital. Various surgical devices and systems are utilized in performance of a surgical procedure. In the digital and information age, medical systems and facilities are often slower to implement systems or procedures utilizing newer and improved technologies due to patient safety and a general desire for maintaining traditional practices. The accuracy with which the patient biomarkers are measured may be influenced by various factors that may lead to inaccurate measurement of the biomarkers. Mechanisms are needed to identify and handle such factors.

### SUMMARY

Surgical systems, surgical instrumentalities, and surgical methods described herein may allow the healthcare professionals (HCPs) (e.g., surgeons) to be notified about leakage current in surgical devices. The ability of HCPs to be notified about the leakage current may prevent HCPs from using the incorrect patient biomarkers readings when a leakage current occurs. Instead, HCPs may wait until the leakage current is corrected or compensated for before using the patient biomarkers for patient monitoring. This may prevent fewer patient monitoring errors and better surgical outcomes.

In examples, an electronic station within a surgical system may include a communications interface configured to connect to a plurality of patient monitoring devices and a plurality of energy devices. The electronic station may receive a signal from one or more of the plurality of energy devices. The signal may notify the electronic station of a leakage current associated with a patient monitoring device of the plurality of patient monitoring devices. The electronic station may determine to adjust the connection with the patient monitoring device to compensate for the leakage current associated with the patient monitoring device based on the received signal. The electronic station may perform the adjustment to the connection with the patient monitoring device to compensate for the leakage current associated with the patient monitoring device.

In examples, the adjustment of the connection with the patient monitoring device may include isolating the connection with the plurality of patient monitoring devices. In examples, the adjustment of the connection with the patient monitoring device may include sending a simulated signal to the patient monitoring device to compensate for the leakage current. In examples, the electronic station may send a notification to the patient monitoring device. The notification may notify the patient monitoring device of the leakage current. The patient monitoring device (e.g., a blood sensing system, a heart rate monitoring system, a skin conductance sensing system, etc.) may detect a patient biomarker (e.g., blood pressure, heart rate, skin conductance, etc.). The leakage current may cause incorrect readings of the patient biomarker associated with the patient monitoring device. The notification may further notify the patient monitoring device of the incorrect readings of the patient biomarker. The adjustment to the connection with the patient monitoring device that compensates for the leakage current may correct the incorrect readings of the patient biomarker.

In examples, an electronic station within a surgical system may include a communications interface configured to connect to a plurality of patient monitoring devices. The electronic station may detect a leakage current associated with a patient monitoring device of the plurality of patient monitoring devices. The electronic station may determine a mechanism to compensate for the leakage current associated with the patient monitoring device. The electronic station may perform the mechanism to compensate for the leakage current associated with the patient monitoring device.

In examples, the determined and performed mechanism to compensate for the leakage current may be to generate a simulated signal for the patient monitoring device. In examples, the determined and performed mechanism to compensate for the leakage current may be to isolate the patient monitoring device. In examples, the electronic station may send a notification to the patient monitoring device. The notification may notify the patient monitoring device of the leakage current. The patient monitoring device (e.g., a blood sensing system, a heart rate monitoring system, a skin conductance sensing system, etc.) may detect a patient biomarker (e.g., blood pressure, heart rate, skin conductance, etc.). The leakage current may cause incorrect readings of the patient biomarker associated with the patient monitoring device. The notification may further notify the patient monitoring device of the incorrect readings of the patient biomarker. The performed mechanism to compensate for the leakage current may correct the incorrect readings of the patient biomarker.

### BRIEF DESCRIPTION OF THE DRAWINGS

FIG. 1 illustrates a block diagram of a computer-implemented surgical system.

FIG. 2 illustrates an example surgical system in a surgical operating room.

FIG. 3 illustrates an example surgical hub paired with various systems.

FIG. 4 illustrates an example of a situationally aware surgical system.

FIG. 5 illustrates an example surgical system that may include a surgical instrument.

FIG. 6 illustrates an example of interactions within the surgical system.

FIG. 7 illustrates an example of a surgical system including an electronic station detecting a leakage current associated with one or more patient monitoring devices.

FIG. 8 illustrates an example of a surgical system including an electronic station receiving a signal from one or more energy device(s) when a leakage current is detected.

FIG. 9 illustrates an example of a simulated signal of a patient monitoring device after a leakage current is detected.

FIG. 10 illustrates an example of isolating a patient monitoring device after a leakage current is detected.

FIG. 11 illustrates an example of a surgical system with the surgical devices driven to a common equipotential.

### DETAILED DESCRIPTION

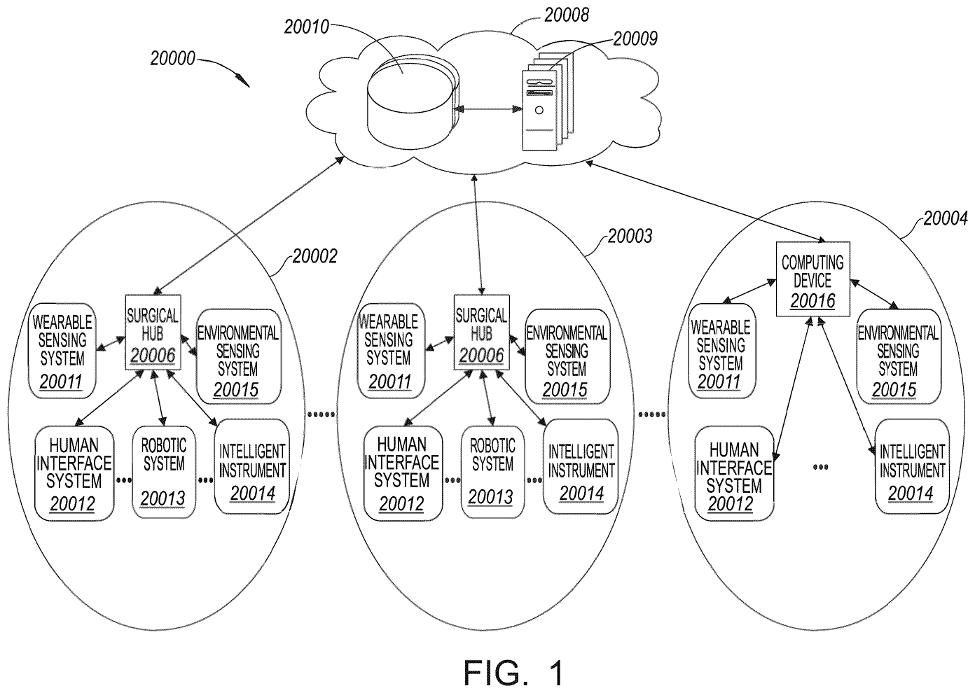

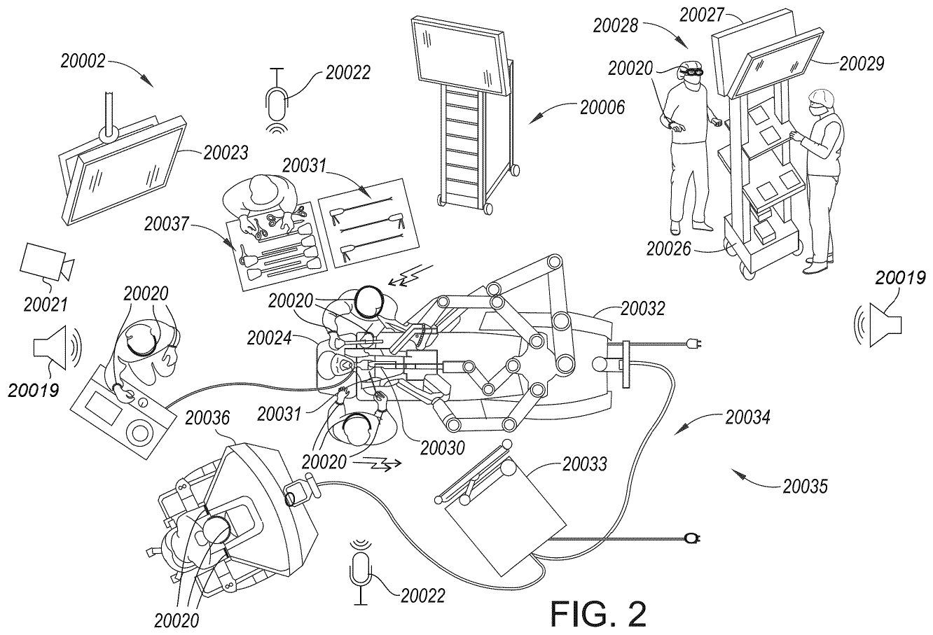

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

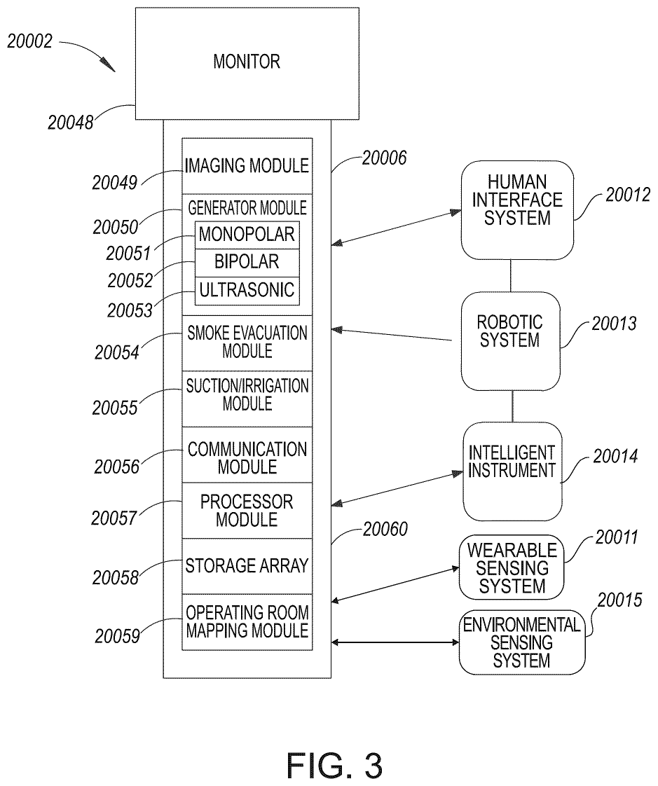

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

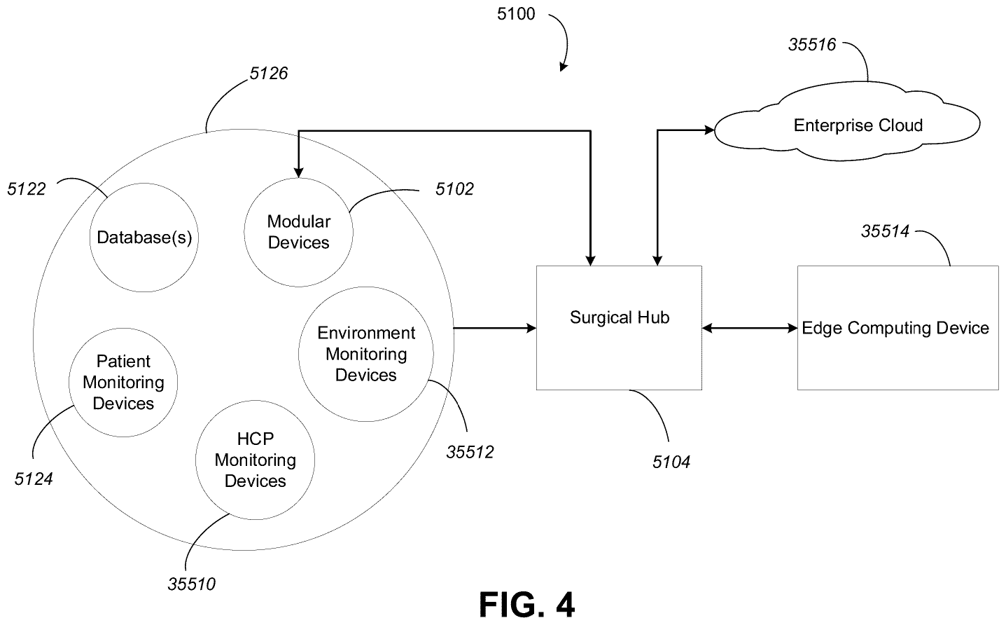

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

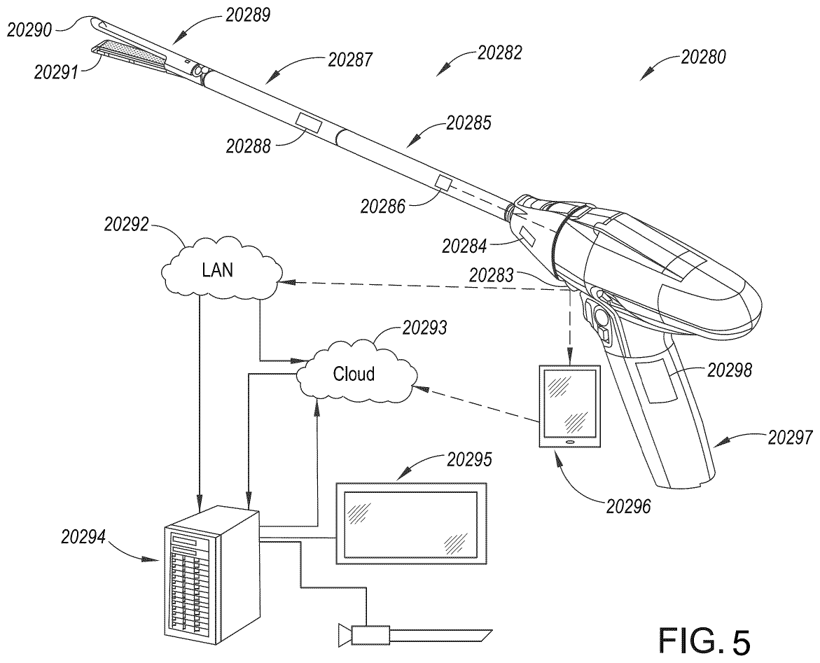

FIG. 5 illustrates an example surgical system 20280 that may include a surgical instrument 20282. The surgical instrument 20282 may correspond with the first surgical device described below. The surgical instrument 20282 can be in communication with a console 20294 and/or a portable device 20296 through a local area network 20292 and/or a cloud network 20293 via a wired and/or wireless connection. The console 20294 and the portable device 20296 may be any suitable computing device. Surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreens, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It will be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (i.e., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

HCPs (e.g., surgeons) may often rely on accurate readings of patient biomarkers from patient monitoring devices for precise patient monitoring. These patient biomarkers may be related to important measurements of patient health (e.g., heart rate, blood sugar, blood pressure). Any miscalculations of these measurements can lead to untreated health conditions that could be detrimental to short and long term patient health. Accurate readings from the patient monitoring devices may be important for avoiding any of these detrimental miscalculations. A proper amount of current flowing across the patient monitoring devices and the surgical system may be one important aspect for achieving accurate readings of patient biomarkers. If any excess current (e.g., leakage current) flows into one or more of the patient monitoring devices, miscalculations may occur. As such, preventing leakage current from entering into the patient monitoring devices may be critical for patient monitoring.

To avoid the detrimental effects of leakage current leading to miscalculations, an electronic station may be configured to detect a leakage current entering into the one or more patient monitoring devices. The electronic station may (e.g., may then) be configured to send a notification to the one or more patient monitoring devices of the leakage current. This may help to notify the HCPs (e.g., surgeons) that the current patient biomarkers measured by the patient monitoring devices may be incorrect, and to not rely on these current measurements for patient monitoring. In examples, the electronic station may (e.g., may also) send a simulated signal to the one or more patient monitoring devices. The simulated signal may compensate for the leakage current, offsetting its effects and leading to accurate biomarker measurements. As such, HCPs may be notified if a leakage current occurs and if a leakage current has been offset, ensuring the confidence of using the patient monitoring devices for monitoring patient biomarkers. This may lead to fewer instances of using incorrect patient biomarker readings for patient monitoring, ultimately leading to better patient healthcare outcomes.

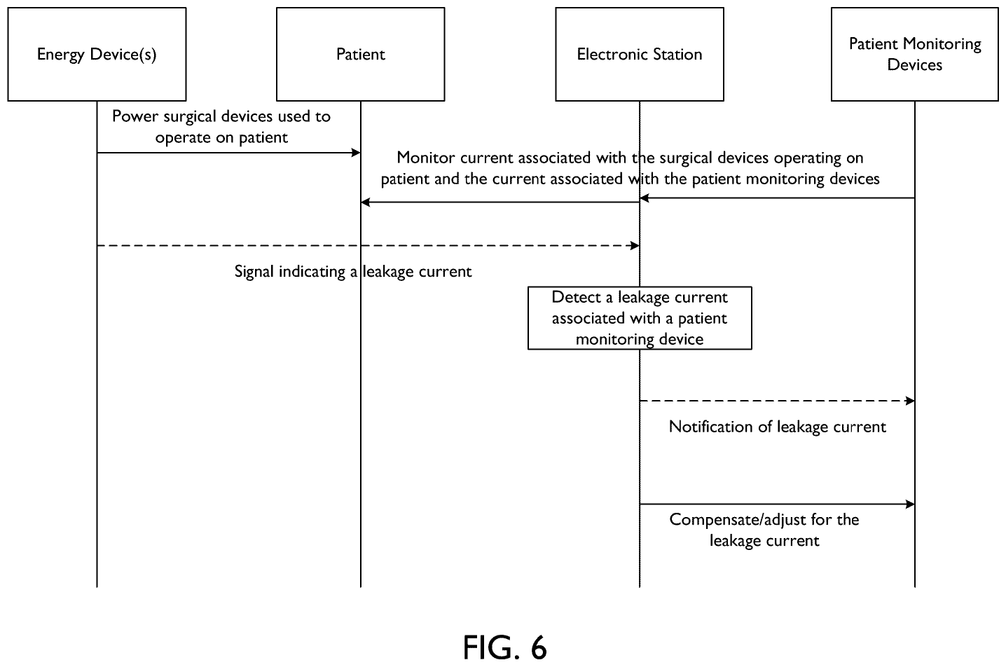

FIG. 6 illustrates an example of interactions within the surgical system. As shown in FIG. 6, the surgical systems may include energy device(s), a patient, an electronic station (e.g., I/O station), and patient monitoring devices. The energy device(s) (e.g., generator(s)) may power the surgical systems used to operate on the patient. In examples, the electronic station may monitor the current associated with the surgical devices operating on a patient and the current associated with the patient monitoring devices. In examples, the electronic station may monitor the current associated with the patient itself. In examples, the energy device(s) within the surgical system may send a signal to the electronic station indicating a leakage current. The electronic station may detect a leakage current associated with at least one of the patient monitoring devices and/or patient. In examples, the electronic station may send a notification of the leakage current to the patient monitoring devices. The electronic station may compensate or adjust for the leakage current.

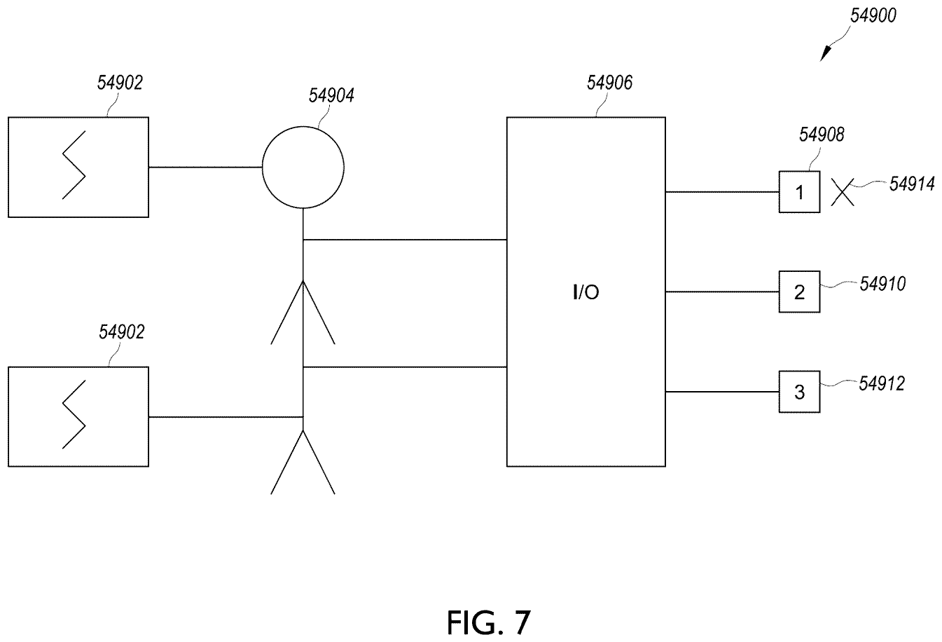

FIG. 7 illustrates an example of a surgical system 54900 including an electronic station 54906 detecting a leakage current associated with one or more monitoring devices 54908, 54910, 54912. The electronic station 54906 may include a communications interface configured to connect to the patient monitoring devices 54908, 54910, 54912. In examples, the electronic station 54906 may be connected with the patient monitoring devices 54908, 54910, 54912 via a wireless connection. In examples, the electronic station 54906 may be connected with the patient monitoring devices 54908, 54910, 54912 via a wired connection. The electronic station 54902 may be an input/output (I/O) device. In examples, the electronic station 54902 may include an ammeter to measure the current through the patient monitoring devices 54908, 54910, 54912. In examples, the electronic station may include sensors to measure current.

The patient monitoring devices 54908, 54910, 54912 may include devices and/or systems that are configured to detect respective patient biomarkers. Examples of patient biomarkers and measurement of such patient biomarkers may be found in application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety. In examples, the patient monitoring devices 54908, 54910, 54912 may include a blood pressure sensing system. The blood pressure sensing system may measure a patient's blood pressure (e.g., the patient 54904). In examples, the patient monitoring devices 54908, 54910, 54912 may include an electrocardiography (EKG) monitor. The EKG monitor may measure a patient's heart rate. In examples, the patient monitoring devices 54908, 54910, 54912 may include a lactate sensing system. The lactate sensing system may employ electrochemical biosensors to measure a patient's sweat lactate levels. In examples, the patient monitoring devices 54908, 54910, 54912 may measure a patient's peripheral capillary oxygen saturation. In examples, the patient monitoring devices 54908, 54910, 54912 may measure a patient's VO2 max (e.g., the body's oxygen consumption ability).

In examples, the patient monitoring devices 54908, 54910, 54912 may include a heart rate sensing system. The heart rate sensing system may measure a patient's heart rate variability. In examples, the patient monitoring devices 54908, 54910, 54912 may include a skin conductance sensing system. The skin conductance sensing system may measure a patient's skin conductance level. In examples, the patient monitoring devices 54908, 54910, 54912 may include a GI motility sensing system. The GI motility sensing system may measure a patient's gastric, small bowel, large bowel, and/or colonic transit times. In examples, the patient monitoring devices 54908, 54910, 54912 may measure a patient's skin perfusion pressure.

In examples, the patient monitoring devices 54908, 54910, 54912 may include a tissue perfusion pressure sensing system. The tissue perfusion pressure sensing system may measure a patient's blood volume changes. In examples, the patient monitoring devices 54908, 54910, 54912 may include a hydration state sensing system. The hydration state sensing system may measure a patient's water content level in the blood. In examples, the patient monitoring devices 54908, 54910, 54912 may include an oxygen saturation system. The oxygen saturation system may measure oxygen saturation. In examples, the patient monitoring devices 54908, 54910, 54912 may include an edema sensing system. In examples, the patient monitoring devices 54908, 54910, 54912 may include electrochemical biosensors. The electrochemical biosensors may measure a patient's sweat lactate levels. In examples, the patient monitoring devices 54908, 54910, 54912 may include a respiration rate monitor. The respiration rate monitor may measure a patient's number of breaths per minute. In examples, the patient monitoring devices 54908, 54910, 54912 may include measurements related to hemostasis related biomarkers. In examples, the patient monitoring devices 54908, 54910, 54912 may include a coagulation sensing system. The coagulation sensing system may measure a patient's blood coagulation status.

The electronic station 54906 may detect a leakage current associated with one or more of the patient monitoring devices 54908, 54910, 54912 (e.g., the patient monitoring device 54908 as shown in FIG. 7). In examples, the leakage current may be detected by an ammeter in the electronic station 54906. In examples, the leakage current may be detected by sensors within the electronic station 54906. The leakage current may cause incorrect readings of the patient biomarkers associated with one or more of the patient monitoring devices 54908, 54910, 54912. The incorrect readings may lead to incorrect diagnostics for HCPs and patients, leading to worse healthcare outcomes. In examples, the electronic station may include sensors to communicate with the one or more patient monitoring devices 54908, 54910, 54912 that are monitoring the biomarkers of the patient 54904. The patient monitoring devices 54908, 54910, 54912 may be connected to or may be worn by the patient 54904.

In examples, the leakage current may lead to incorrect blood pressure level readings. In examples, the leakage current may lead to incorrect heart rate readings. In examples, the leakage current may lead to incorrect sweat lactate level readings. In examples, the leakage current may lead to incorrect oxygen saturation level readings. In examples, the leakage current may lead to incorrect oxygen consumption readings. In examples, the leakage current may lead to incorrect heart rate variability readings. In examples, the leakage current may lead to incorrect skin conductance level readings. In examples, the leakage current may lead to incorrect gastric, small bowel, large bowel, and/or colonic transit time readings. In examples, the leakage current may lead to incorrect skin perfusion pressure readings. In examples, the leakage current may lead to incorrect readings of water content level in the blood. In examples, the leakage current may lead to incorrect readings of the number of breaths per minute. In examples, the leakage current leakage current may lead to incorrect readings of blood coagulation status readings.

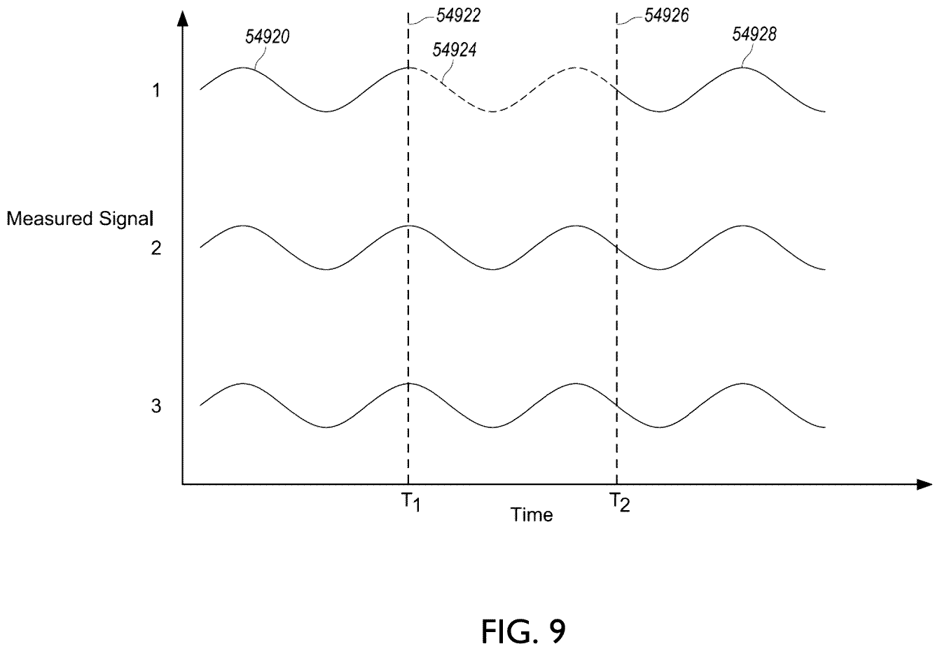

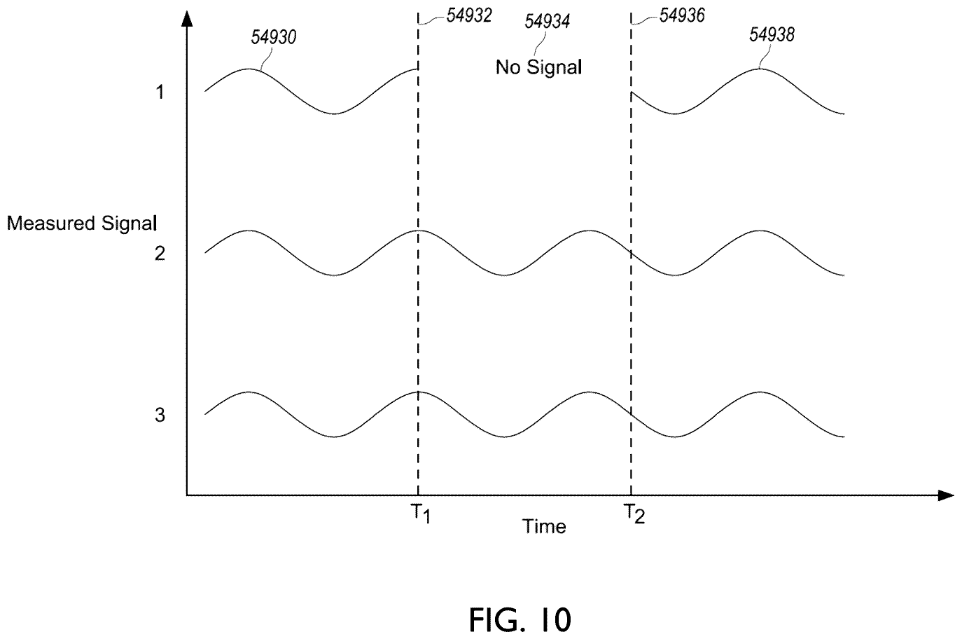

The electronic station 54906 may determine a mechanism to compensate for the leakage current 54914 associated with one or more patient monitoring devices 54908, 54910, 54912 (e.g., the patient monitoring device 54908 as shown in FIG. 7). In examples, the electronic station 54906 may determine to simulate a signal (e.g., as shown in FIG. 9) for the one or more patient monitoring devices 54908, 54910, 54912 (e.g., the patient monitoring device 54908 as shown in FIG. 7) to compensate for the leakage current. The simulated signal may have characteristics that would be obvious to the HCPs. The simulated signal may be designed to specifically not induce an alarm in the monitoring system while protecting the monitor from the leakage current from the activation of energy. In examples, the electronic station 54906 may determine to isolate (e.g., as shown in FIG. 10) the one or more patient monitoring devices 54908, 54910, 54912 (e.g., the patient monitoring device 54908 as shown in FIG. 7) to compensate for the leakage current.

The electronic station 54906 may perform the mechanism to compensate for the leakage current 54914 associated with one or more patient monitoring devices 54908, 54910, 54912 (e.g., the patient monitoring device 54908 as shown in FIG. 7). In examples, the electronic station 54906 may send the simulated signal to the one or more patient monitoring devices 54908, 54910, 54912 (e.g., the patient monitoring device 54908 as shown in FIG. 7) to compensate for the leakage current. In examples, the electronic station 54906 may isolate the one or more patient monitoring devices 54908, 54910, 54912 (e.g., the patient monitoring device 54908 as shown in FIG. 7) to compensate for the leakage current.

The determined mechanism may compensate for the leakage current and may correct the incorrect readings of the patient biomarkers. In examples, simulating the signal for or isolating the one or patient monitoring devices 54908, 54910, 54912 may compensate for incorrect blood pressure level readings. In examples, simulating the signal for or isolating the one or patient monitoring devices 54908, 54910, 54912 may compensate for incorrect heart rate readings. In examples, simulating the signal for or isolating the one or patient monitoring devices 54908, 54910, 54912 may compensate for incorrect sweat lactate level readings. In examples, simulating the signal for or isolating the one or patient monitoring devices 54908, 54910, 54912 may compensate for incorrect oxygen saturation level readings. In examples, simulating the signal for or isolating the one or patient monitoring devices 54908, 54910, 54912 may compensate for incorrect oxygen consumption readings. In examples, simulating the signal for or isolating the one or patient monitoring devices 54908, 54910, 54912 may compensate for incorrect heart rate variability readings. In examples, simulating the signal for or isolating the one or patient monitoring devices 54908, 54910, 54912 may compensate for incorrect skin conductance level readings. In examples, simulating the signal for or isolating the one or patient monitoring devices 54908, 54910, 54912 may compensate for incorrect gastric, small bowel, large bowel, and/or colonic transit time readings. In examples, simulating the signal for or isolating the one or patient monitoring devices 54908, 54910, 54912 may compensate for incorrect skin perfusion pressure readings. In examples, simulating the signal for or isolating the one or patient monitoring devices 54908, 54910, 54912 may compensate for incorrect readings of water content level in the blood. In examples, simulating the signal for or isolating the one or patient monitoring devices 54908, 54910, 54912 may compensate for incorrect readings of the number of breaths per minute. In examples, simulating the signal for or isolating the one or patient monitoring devices 54908, 54910, 54912 may compensate for incorrect readings of blood coagulation status readings.

In examples, the electronic station 54906 may send a notification to one or more of the patient monitoring devices 54908, 54910, 54912. The notification may notify the one or more patient monitoring devices 54908, 54910, 54912 of the leakage current. The notification may be in the form of an alert or an alarm that would notify the HCPs of the leakage current, allowing the HCPs to adjust the health care monitoring for the patient accordingly.

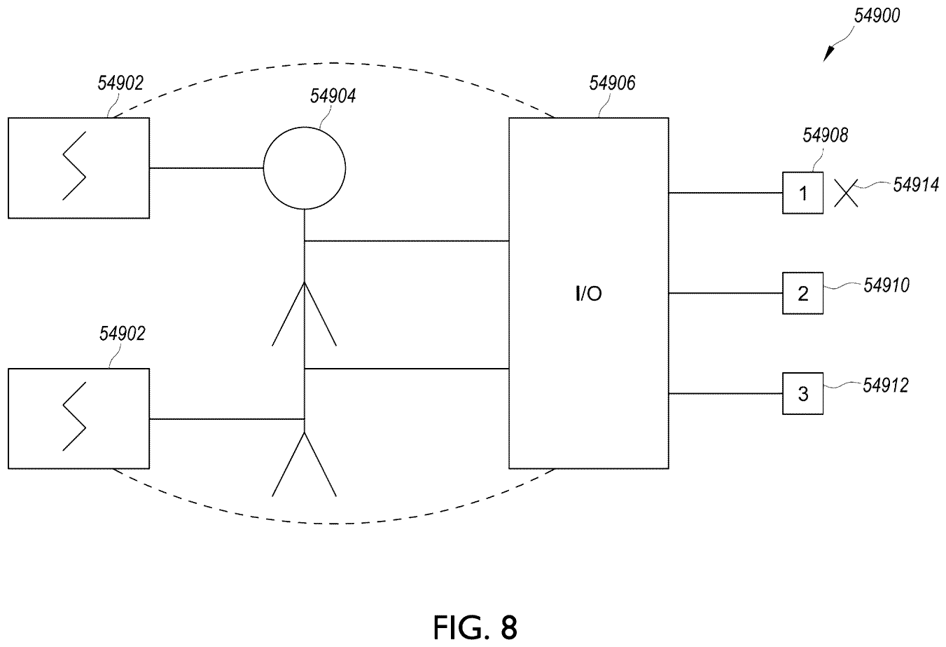

FIG. 8 illustrates an example of a surgical system including an electronic station 54906 receiving a signal from one or more electronic device(s) 54902 when a leakage current is detected. The electronic station 54906 may include a communications interface configured to communicate with the patient monitoring devices 54908, 54910, 54912 and one or more electronic device(s) 54902. As shown in FIG. 8, the patient monitoring device 54908 is indicated by (1) as shown in FIGS. 8-10, the patient monitoring device 54910 is indicated by (2) as shown in FIGS. 8-10, and the patient monitoring device 54912 is indicated by (3) as shown in FIGS. 8-10. In examples, the electronic station 54906 may be connected with the patient monitoring devices 54908, 54910, 54912 and the one or more energy device(s) 54902 via a wireless connection. In examples, the electronic station 54906 may be connected with the patient monitoring devices 54908, 54910, 54912 and the one or more energy device(s) 54902 via a wired connection.

The electronic station 54902 may be an input/output (I/O) device. In examples, the electronic station 54902 may include an ammeter to measure the current through the patient monitoring devices 54908, 54910, 54912. In examples, the electronic station may include sensors to measure current. The one or more energy device(s) 54902 may be configured to drive signals to the electronic station 54906. In examples, the drive signal may output an ultrasonic drive signal to the electronic station 54906. In examples, the drive signal may output an RF electrosurgical drive signal to the electronic station 54906. Additional details of one or more energy device(s) 54902 are described in U.S. Pat. No. 9,060,775, titled SURGICAL GENERATOR FOR ULTRASONIC AND ELECTROSURGICAL DEVICES, which issued on Jun. 23, 2015, which is herein incorporated by reference in its entirety.

The patient monitoring devices 54908, 54910, 54912 may include devices and/or systems with sensing systems that are configured to detect respective patient biomarkers. In examples, the patient monitoring devices 54908, 54910, 54912 may include a blood pressure sensing system. The blood pressure sensing system may measure a patient's blood pressure (e.g., the patient 54904). In examples, the patient monitoring devices 54908, 54910, 54912 may include an electrocardiography (EKG) monitor. The EKG monitor may measure a patient's heart rate. In examples, the patient monitoring devices 54908, 54910, 54912 may include a lactate sensing system. The lactate sensing system may employ electrochemical biosensors to measure a patient's sweat lactate levels. In examples, the patient monitoring devices 54908, 54910, 54912 may measure a patient's peripheral capillary oxygen saturation. In examples, the patient monitoring devices 54908, 54910, 54912 may measure a patient's VO2 max (e.g., the body's oxygen consumption ability).

In examples, the patient monitoring devices 54908, 54910, 54912 may include a heart rate sensing system. The heart rate sensing system may measure a patient's heart rate variability. In examples, the patient monitoring devices 54908, 54910, 54912 may include a skin conductance sensing system. The skin conductance sensing system may measure a patient's skin conductance level. In examples, the patient monitoring devices 54908, 54910, 54912 may include a GI motility sensing system. The GI motility sensing system may measure a patient's gastric, small bowel, large bowel, and/or colonic transit times. In examples, the patient monitoring devices 54908, 54910, 54912 may measure a patient's skin perfusion pressure.

In examples, the patient monitoring devices 54908, 54910, 54912 may include a tissue perfusion pressure sensing system. The tissue perfusion pressure sensing system may measure a patient's blood volume changes. In examples, the patient monitoring devices 54908, 54910, 54912 may include a hydration state sensing system. The hydration state sensing system may measure a patient's water content level in the blood. In examples, the patient monitoring devices 54908, 54910, 54912 may include an oxygen saturation system. The oxygen saturation system may measure oxygen saturation. In examples, the patient monitoring devices 54908, 54910, 54912 may include an edema sensing system. In examples, the patient monitoring devices 54908, 54910, 54912 may include electrochemical biosensors. The electrochemical biosensors may measure a patient's sweat lactate levels. In examples, the patient monitoring devices 54908, 54910, 54912 may include a respiration rate monitor. The respiration rate monitor may measure a patient's number of breaths per minute. In examples, the patient monitoring devices 54908, 54910, 54912 may include measurements related to hemostasis related biomarkers. In examples, the patient monitoring devices 54908, 54910, 54912 may include a coagulation sensing system. The coagulation sensing system may measure a patient's blood coagulation status.

The electronic station 54906 may receive a signal from one or more energy device(s) 54902. The signal may notify the electronic station 54906 of a leakage current 54914 associated with one or more patient monitoring devices 54908, 54910, 54912 (e.g., the patient monitoring device 54908 as shown in FIG. 8). In examples, the leakage current may be detected by the one or more energy device(s) 54902 by the use of a blanking circuit integrated into the one or more energy device(s) 54902. The leakage current may cause incorrect readings of the patient biomarkers associated with one or more of the patient monitoring devices 54908, 54910, 54912. For example, the leakage current may cause interference with an electrochemical or an electromagnetic biosensor measuring patient's lactate levels or skin conductance. The incorrect readings may lead to incorrect diagnostics for HCPs and patients, leading to worse healthcare outcomes. In examples, the electronic station may include sensors to communicate with the one or more patient monitoring devices 54908, 54910, 54912 that are monitoring the biomarkers of the patient 54904. The patient monitoring devices 54908, 54910, 54912 may be connected to or may be worn by the patient 54904.

In examples, the leakage current may cause interference with a biosensor measuring a patient's blood pressure, which may lead to incorrect blood pressure level readings. In examples, the leakage current may cause interference with a biosensor measuring a patient's heart rate, which may lead to incorrect heart rate readings. In examples, the leakage current may cause interference with a biosensor measuring a patient's sweat lactate level, which may lead to incorrect sweat lactate level readings. In examples, the leakage current may cause interference with a biosensor measuring a patient's oxygen saturation level, which may lead to incorrect oxygen saturation level readings. In examples, the leakage current may cause interference with a biosensor measuring a patient's oxygen consumption, which may lead to incorrect oxygen consumption readings. In examples, the leakage current may cause interference with a biosensor measuring a patient's heart rate variability, which may lead to incorrect heart rate variability readings. In examples, the leakage current may cause interference with a biosensor measuring a patient's skin conductance level, which may lead to incorrect skin conductance level readings. In examples, the leakage current may lead to incorrect gastric, small bowel, large bowel, and/or colonic transit time readings. In examples, the leakage current may cause interference with a biosensor measuring a patient's skin perfusion pressure, which may lead to incorrect skin perfusion pressure readings. In examples, the leakage current may cause interference with a biosensor measuring a patient's water content level in the blood, which may lead to incorrect readings of water content level in the blood. In examples, the leakage current may cause interference with a biosensor measuring a patient's number of breaths per minute, which may lead to incorrect readings of the number of breaths per minute. In examples, the leakage current leakage current may cause interference with a biosensor measuring a patient's blood coagulation status, which may lead to incorrect readings of blood coagulation status.

The electronic station 54906 may determine to adjust the connection with the one of more patient monitoring devices 54908, 54910, 54912 to compensate for the leakage current 54914 associated with one or more patient monitoring devices 54908, 54910, 54912 (e.g., the patient monitoring device 54908 as shown in FIG. 8) based on the received signal from the one or more energy device(s) 54902. In examples, the electronic station 54906 may isolate the connection from the one or more patient monitoring devices 54908, 54910, 54912 (e.g., the patient monitoring device 54908 as shown in FIG. 8) to compensate for the leakage current 54914. In examples, the electronic station 54906 may short the connection with the one or more patient monitoring devices 54908, 54910, 54912 (e.g., the patient monitoring device 54908 as shown in FIG. 8) to compensate for the leakage current 54914 until the leakage current 54914 is eliminated. In examples, the electronic station 54906 may send a simulated signal to the one or more patient monitoring devices 54908, 54910, 54912 (e.g., the patient monitoring device 54908 as shown in FIG. 8) to compensate for the leakage current 54914.

The electronic station 54906 may perform the mechanism to compensate for the leakage current 54914 associated with one or more patient monitoring devices 54908, 54910, 54912 (e.g., the patient monitoring device 54908 as shown in FIG. 8). In examples, the electronic station 54906 may send the simulated signal to the one or more patient monitoring devices 54908, 54910, 54912 (e.g., the patient monitoring device 54908 as shown in FIG. 8) to compensate for the leakage current. In examples, the electronic station 54906 may isolate the one or more patient monitoring devices 54908, 54910, 54912 (e.g., the patient monitoring device 54908 as shown in FIG. 8) to compensate for the leakage current.

The determined mechanism may compensate for the leakage current and may correct the incorrect readings of the patient biomarkers. In examples, simulating the signal for or isolating the one or patient monitoring devices 54908, 54910, 54912 may compensate for incorrect blood pressure level readings. In examples, simulating the signal for or isolating the one or patient monitoring devices 54908, 54910, 54912 may compensate for incorrect heart rate readings. In examples, simulating the signal for or isolating the one or patient monitoring devices 54908, 54910, 54912 may compensate for incorrect sweat lactate level readings. In examples, simulating the signal for or isolating the one or patient monitoring devices 54908, 54910, 54912 may compensate for incorrect oxygen saturation level readings. In examples, simulating the signal for or isolating the one or patient monitoring devices 54908, 54910, 54912 may compensate for incorrect oxygen consumption readings. In examples, simulating the signal for or isolating the one or patient monitoring devices 54908, 54910, 54912 may compensate for incorrect heart rate variability readings. In examples, simulating the signal for or isolating the one or patient monitoring devices 54908, 54910, 54912 may compensate for incorrect skin conductance level readings. In examples, simulating the signal for or isolating the one or patient monitoring devices 54908, 54910, 54912 may compensate for incorrect gastric, small bowel, large bowel, and/or colonic transit time readings. In examples, simulating the signal for or isolating the one or patient monitoring devices 54908, 54910, 54912 may compensate for incorrect skin perfusion pressure readings. In examples, simulating the signal for or isolating the one or patient monitoring devices 54908, 54910, 54912 may compensate for incorrect readings of water content level in the blood. In examples, simulating the signal for or isolating the one or patient monitoring devices 54908, 54910, 54912 may compensate for incorrect readings of the number of breaths per minute. In examples, simulating the signal for or isolating the one or patient monitoring devices 54908, 54910, 54912 may compensate for incorrect readings of blood coagulation status.

In examples, the electronic station 54906 may send a notification to one or more of the patient monitoring devices 54908, 54910, 54912. The notification may notify the one or more patient monitoring devices 54908, 54910, 54912 of the leakage current. The notification may be in the form of an alert or an alarm that would notify the HCPs of the leakage current, allowing the HCPs to adjust the health care monitoring for the patient accordingly.

FIG. 9 illustrates an example of a simulated signal of a patient monitoring device while a leakage current is being detected. The patient monitoring device(s) may use their normal measured signals (e.g., such as a normal measured signal 54920 as shown in FIG. 9) before a leakage current is detected (e.g., before T1 as shown in FIG. 9). As described herein, when a leakage current is detected at 54922 (T1) in the one or more patient monitoring devices 54908, 54910, 54912 by the electronic station 54906 or the electronic station 54906 receives a signal from the one or more energy device(s) 54902 that indicates a leakage current in the one or more patient monitoring devices 54908, 54910, 54912, the electronic station 54906 may send a simulated signal 54924 (as shown by the dashed line in FIG. 9) to the patient monitoring device(s) (e.g., as shown by (1) in FIG. 9) experiencing the leakage current. After the time 54922 (T1) the leakage current is detected, the patient monitoring device(s) (e.g., as shown by (1) in FIG. 9) may use the simulated signal 54924 to compensate for the leakage current (e.g., as shown by the dashed line in FIG. 9). The rest of the patient monitoring device(s) (e.g., as shown by (2) and (3) in FIG. 9) may continue operating with their respective normal measured signals. After the leakage is eliminated at 54926 (T2), the patient monitoring device(s) experiencing the leakage current (e.g., as shown by (1) in FIG. 9) may stop using the simulated signal 54924 and use their normal measured signal(s) again (e.g., a normal measured signal 54928 as shown in FIG. 9).

FIG. 10 illustrates an example of isolating a patient monitoring device while a leakage current is being detected. The patient monitoring device(s) may use their normal measured signals (e.g., such as a normal measured signal 54930 as shown in FIG. 10) before a leakage current is detected (e.g., before T1 as shown in FIG. 10). As described herein, when a leakage current is detected at 54932 (T1) in the one or more patient monitoring devices 54908, 54910, 54912 by the electronic station 54906 or the electronic station 54906 receives a signal from the one or more energy device(s) 54902 that indicates a leakage current in the one or more patient monitoring devices 54908, 54910, 54912, the electronic station 54906 may isolate the patient monitoring device(s) (e.g., as shown by (1) in FIG. 10) experiencing the leakage current by providing no signal to the patient monitoring device at 54934 as shown in FIG. 10. After the time 54932 (T1) the leakage current is detected, the patient monitoring device(s) (e.g., as shown by 1 in FIG. 10) may receive no signal at 54934 to compensate for the leakage current. The rest of the patient monitoring device(s) (e.g., as shown by (2) and (3) in FIG. 10) may continue operating with their respective normal measured signals. After the leakage current is eliminated at 54936 (T2), the patient monitoring device(s) experiencing the leakage current (e.g., as shown by (1) in FIG. 10) may use their normal measured signal(s) again (e.g., a normal measured signal 54938 as shown in FIG. 10).

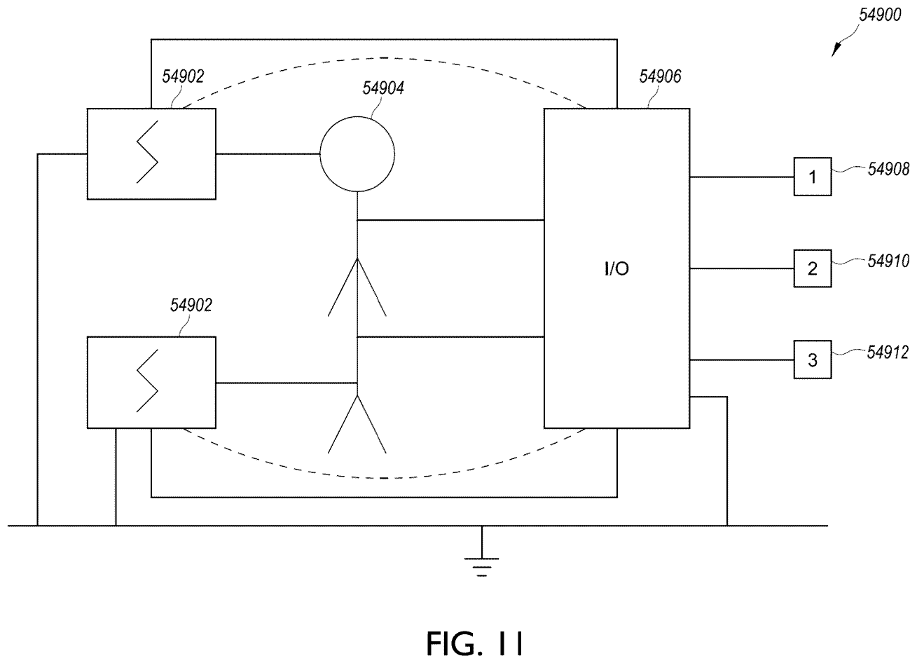

FIG. 11 illustrates an example of a surgical system with the surgical devices driven to a common equipotential. As shown in FIG. 11, the one or more energy device(s) 54902 and one or more patient monitoring devices 54908, 54910, 54912 may be driven to a common equipotential. In examples, the common floating potential may be earth ground. The ground paths or patient potential between the energy device(s) 54902 and patient monitoring devices 54908, 54910, 54912 may be synchronized. The return pads may be coupled and therefore return path of the energy device(s) 54902 may be grounded. In examples, there could be a controlled electrical potential that the patient and all equipment tied to the patient could all couple to.

The energy device(s) 54902 may have an integrated or isolated electronic station 54906 that is placed between the patient 54904 and the patient monitoring devices 54908, 54910, 54912. With the activation of energy, the electronic station 54906 may isolate all systems coupled thru the electronic station 54906. In order to prevent the monitor from throwing a code due to loss of signal the, the electronic station 54906 may either send a notification signal to the monitoring device or a simulated signal that would satisfy the monitoring system's input requirements but would not originate from the patient. This artificial signal may have characteristics that would be obvious to the surgeon but designed to specifically not induce an alarm in the monitoring system while protecting the monitor from the leakage current from the activation of energy.

In examples, the one or more energy device(s) 54902 may include a blanking circuit (e.g., the blanking circuit may be integrated into some advanced generators). The one or more energy device(s) 54902 may have an output signal that may be generated on a separate channel just prior to or in combination with the advanced energy application (e.g., in this case). This signal may be received by another system (e.g., the electronic station 54906) that has the built-in capability to use the signal as a means for either ignoring or isolating the connection of its electrical connection to the patient during this application.

In examples, if any of the measurement leads sense a potential shift between their leads, the leads may be actively shunted, which may allow the potential between the leads to dissipate before the shunting is broken and the system can again be used at the equipotential level to the other connected devices to the patient 54904. The active shunting system may drive a detected non-equipotential level to a common level by actively shorting the leads until the potential variance is gone.

What is claimed:

1. An electronic station within a surgical system, comprising:
a communications interface configured to connect to a plurality of patient monitoring devices and a plurality of energy devices; and
a processor configured to:
receive a signal from one or more of the plurality of energy devices, wherein the signal notifies the electronic station of a leakage current associated with a patient monitoring device of the plurality of patient monitoring devices;
determine to adjust the connection with the patient monitoring device to compensate for the leakage current associated with the patient monitoring device based on the received signal; and
perform the adjustment to the connection with the patient monitoring device to compensate for the leakage current associated with the patient monitoring device.

2. The electronic station of claim 1, wherein the adjustment of the connection with the patient monitoring device comprises isolating the connection with the plurality of patient monitoring devices.

3. The electronic station of claim 1, wherein the adjustment of the connection with the patient monitoring device comprises sending a simulated signal to the patient monitoring device to compensate for the leakage current.

4. The electronic station of claim 1, wherein the processor is further configured to:
send a notification to the patient monitoring device, wherein the notification notifies the patient monitoring device of the leakage current.

5. The electronic station of claim 4, wherein:
the patient monitoring device is configured to detect a patient biomarker,
the leakage current causes incorrect readings of the patient biomarker associated with the patient monitoring device,
the notification further notifies the patient monitoring device of the incorrect readings of the patient biomarker, and
the adjustment to the connection with the patient monitoring device to compensate for the leakage current corrects the incorrect readings of the patient biomarker.

6. The electronic station of claim 5, wherein:
the patient monitoring device is a blood pressure sensing system,
the patient biomarker is blood pressure,
the notification further notifies the blood pressure sensing system of the incorrect readings of the blood pressure, and
the adjustment to the connection with the patient monitoring device to compensate for the leakage currents correct the incorrect readings of the blood pressure.

7. The electronic station of claim 5, wherein:
the patient monitoring device is a heart rate monitoring system,
the patient biomarker is heart rate,
the notification further notifies the heart rate monitoring system of the incorrect readings of the heart rate, and
the adjustment to the connection with the patient monitoring device to compensate for the leakage current corrects the incorrect readings of the heart rate.

8. The electronic station of claim 5, wherein:
the patient monitoring device is a skin conductance sensing system,
the patient biomarker is skin conductance,
the notification further notifies the skin conductance sensing system of the incorrect readings of the skin conductance, and
the adjustment to the connection with the patient monitoring device to compensate for the leakage current corrects the incorrect readings of the skin conductance.

9. An electronic station within a surgical system, comprising:
a communications interface configured to connect to a plurality of patient monitoring devices; and
a processor configured to:
detect a leakage current associated with a patient monitoring device of the plurality of patient monitoring devices;
determine a mechanism to compensate for the leakage current associated with the patient monitoring device; and
perform the mechanism to compensate for the leakage current associated with the patient monitoring device.

10. The electronic station of claim 9, wherein:
the determined mechanism to compensate for the leakage current is to generate a simulated signal for the patient monitoring device, and
the performed mechanism to compensate for the leakage current is to send the simulated signal to the patient monitoring device.

11. The electronic station of claim 9, wherein:
the determined mechanism to compensate for the leakage current is to isolate the patient monitoring device, and
the performed mechanism to compensate for the leakage current is to isolate the patient monitoring device.

12. The electronic station of claim 9, wherein the processor is further configured to:
send a notification to the patient monitoring device, wherein the notification notifies the patient monitoring device of the leakage current.

13. The electronic station of claim 12, wherein:
the patient monitoring device is configured to detect a patient biomarker,
the leakage current causes incorrect readings of the patient biomarker associated with the patient monitoring device,
the notification further notifies the patient monitoring device of the incorrect readings of the patient biomarker, and
the performed mechanism to compensate for the leakage currents corrects the incorrect readings of the patient biomarker.

14. The electronic station of claim 13, wherein:
the patient monitoring device is a blood pressure sensing system,
the patient biomarker is blood pressure,
the notification further notifies the blood pressure sensing system of the incorrect readings of the blood pressure, and
the performed mechanism to compensate for the leakage current corrects the incorrect readings of the blood pressure.

15. The electronic station of claim 13, wherein:
the patient monitoring device is a heart rate monitoring system,
the patient biomarker is heart rate,
the notification further notifies the heart rate monitoring system of the incorrect readings of the heart rate, and
the performed mechanism to compensate for the leakage current corrects the incorrect readings of the heart rate.

16. The electronic station of claim 13, wherein:
the patient monitoring device is a skin conductance sensing system,
the patient biomarker is skin conductance,
the notification further notifies the skin conductance sensing system of the incorrect readings of the skin conductance, and
the performed mechanism to compensate for the leakage current corrects the incorrect readings of the skin conductance.

17. A surgical system, comprising:
a plurality of patient monitoring devices;
a plurality of energy devices;
an electronic station configured to connect to the plurality of energy devices and the plurality of patient monitoring devices, wherein the electronic station comprises a processor configured to:
detect a leakage current associated with a patient monitoring device of the plurality of patient monitoring devices;
determine a mechanism to compensate for the leakage current associated with the patient monitoring device; and
perform the mechanism to compensate for the leakage current associated with the patient monitoring device.

18. The surgical system of claim 17, wherein:
the determined mechanism to compensate for the leakage current is to generate a simulated signal for the patient monitoring device, and
the performed mechanism to compensate for the leakage current is to send the simulated signal to the patient monitoring device.

19. The surgical system of claim 17, wherein:
the determined mechanism to compensate for the leakage current is to isolate the patient monitoring device, and
the performed mechanism to compensate for the leakage current is to isolate the patient monitoring device.

20. The surgical system of claim 17, wherein the processor is further configured to:
send a notification to the patient monitoring device, wherein the notification notifies the patient monitoring device of the leakage current.
